(19) United States
(12) Patent Application Publication
(10) Pub. No.: US 2007/0162154 A1
(43) Pub. Date: Jul. 12, 2007

(54) PROSTHETIC MEMBER ORTHOPEDIC COVER

(76) Inventor: John Scott, Dallas, TX (US)

Correspondence Address: WINSTEAD SECHREST & MINICK P.C., P.O. BOX 50784, DALLAS, TX, 75201, US

(21) Appl. No.: 11/622,438

(22) Filed: Jan. 11, 2007

Related U.S. Application Data
(60) Provisional application No. 60/758,591, filed on Jan. 12, 2006.

(51) Int. Cl. A61F 2/80

(52) U.S. Cl. 623/36

(57) ABSTRACT

An orthopedic cover for a prosthetic member including a hollow flexible body portion, a generally tapered cylindrical body adapted for being received over the prosthetic member, an upper opening having a depth sufficient to allow articulation of a prosthetic knee of the prosthetic member and a width sufficient to allow protection of a mechanical linkage of the prosthetic member, and a lower opening exposing a prosthetic limb outwardly therefrom.

### CROSS-REFERENCE TO RELATED APPLICATIONS

This patent application claims priority from, and incorporates by reference the entire disclosure of, U.S. Provisional Patent Application No. 60/758,591, filed Jan. 12, 2006.

### BACKGROUND OF THE INVENTION

1. Technical Field

The present invention relates to protective prosthetic limb covers and, more particularly, but not by way of limitation, to cosmetic covers for prosthetic joints, particularly prosthetic knee or elbow joints.

2. History of Related Art

The use of prosthetic limbs for amputees spans many centuries. Early versions of the prosthetic limbs were simply immoveable extensions of an existing limb, while modern versions are much more technologically advanced. The modern prosthetic knee joint, for example, may include a plurality of mechanical as well electrical systems for imparting movement similar to that of a normal limb. In order to effect such movement, the mechanical linkage must include a pivotal joint allowing angular movement of an underlying limb region relative to an upper portion attached to the user's limb.

The interconnection between the user's limb and the prosthetic limb is typically provided by a socket. Multiple prosthetic limb designs have been utilized for connecting the prosthetic limb to the socket for affording the user the ability to position the prosthetic limb in such a way as to allow walking, running and even jumping. Modern prosthetic knee joints, for example, facilitate such motion. In some instances, the mechanical linkages that allow the articulation necessary for effective limb movement are partially exposed. The articulation occurs around a prosthetic joint and the linkage, by definition, is partially exposed. In some advanced systems, the functioning of the mechanical linkage is augmented by a battery operated electronic system that provides enhanced movement features. Obviously, the exposed mechanical linkage as well as the potential exposure of the electronic system pose potential problems for the user of the prosthetic knee joint. Dirt, debris and/or water that infiltrates the electro-mechanical system that comprises the prosthetic limb joint of current design can cause severe problems, including malfunctioning. In spite of this fact, the use of the prosthetic limb is typically not limited to clean or dry environments.

One example is the use of prosthetic limbs for athletes. It is very common to see participants in athletic events such as, for example, long distance running, using prosthetic members such as, for example, a prosthetic knee joint. During an outdoor race, wind, rain and/or dirt can be blown into any exposed portion of a prosthesis, including a prosthetic joint. In the event that water improperly penetrates a prosthetic knee joint that is battery operated, the detrimental side effect could leave the athlete stranded. In another example, an individual may use prosthetic members for day to day activity such as, for example, showering and water related activities such as, for example, swimming. Thus, in order to avoid damage to the prosthetic member, the prosthetic member should be protected from water and kept dry. Another example is the use of prosthetic knee joints by individuals skydiving and the like. Reliability is clearly a critical element in the use of a prosthetic knee joint for an individual who is skydiving. The proper functioning of the knee joint in a safe landing is critical. Likewise, the protection of the prosthetic limb linkages is also critical.

Therefore, there is a need for a flexible water-proof cover for a prosthetic knee joint that prevents the prosthetic knee joint from dirt, debris and/or water along with being aesthetically pleasing and inexpensive to manufacture.

### SUMMARY OF THE INVENTION

An orthopedic cover for a prosthetic member including a hollow flexible body portion, a generally tapered cylindrical body adapted for being received over the prosthetic member, an upper opening having a depth sufficient to allow articulation of a prosthetic knee of the prosthetic member and a width sufficient to allow protection of a mechanical linkage of the prosthetic member, and a lower opening exposing a prosthetic limb outwardly therefrom.

An orthopedic cover for a prosthetic member including a generally cylindrical body having a curvilinear shape and adapted for receipt of a prosthetic member therethrough, a slotted opening in an upper end of the cover adapted to protect mechanical elements of the orthopedic member while affording a mechanical linkage portion of the prosthetic member to pass through and permit necessary movement of the prosthetic member, and an opening in a lower end of the cover to allow an end of the prosthetic member pass through.

An orthopedic cover for a prosthetic member of a given shape including a hollow flexible body portion, a configured cylindrical body adapted for being received over the prosthetic member for covering the shape thereof and an upper opening having a depth sufficient to allow articulation of a prosthetic knee of the prosthetic member and a width sufficient to allow protection of a mechanical linkage of the prosthetic member. The cover further includes a lower opening exposing a prosthetic limb outwardly therefrom and a hook and pile surface to facilitate securement of the cover about a housing of the prosthetic member.

### BRIEF DESCRIPTION OF THE DRAWINGS

A more complete understanding of embodiments of the present invention can be achieved by reference to the following Detailed Description when taken in conjunction with the accompanying Drawings wherein:

FIG. 1A illustrates a perspective view of a cover for a prosthetic knee joint in accordance with an embodiment the present invention;

FIG. 1B illustrates a top view of the cover for the prosthetic knee joint;

FIG. 1C illustrates a bottom view of the cover for the prosthetic knee joint;

FIG. 1D illustrates a side view of the cover placed over the prosthetic knee joint;

FIG. 2 illustrates a perspective view of the cover for the prosthetic knee joint of FIG. 1A shown along side the prosthetic knee joint;

FIG. 3 illustrates a perspective view of the cover for the prosthetic knee joint in accordance with an alternate embodiment the present invention;

FIG. 4 illustrates a perspective view of the cover for the prosthetic knee joint in accordance with an embodiment the present invention;

FIG. 5 illustrates a perspective view of the cover for the prosthetic knee joint in accordance with an alternate embodiment the present invention;

FIG. 6 illustrates a perspective view of the cover for the prosthetic knee joint in accordance with an embodiment the present invention; and

FIG. 7 illustrates a perspective view of the cover for the prosthetic knee joint in accordance with yet another alternate embodiment the present invention.

### DETAILED DESCRIPTION OF ILLUSTRATIVE EMBODIMENTS OF THE INVENTION

Embodiment(s) of the invention will now be described more fully with reference to the accompanying Drawings. The invention may, however, be embodied in many different forms and should not be construed as limited to the embodiment(s) set forth herein. The invention should only be considered limited by the claims and the equivalents thereof.

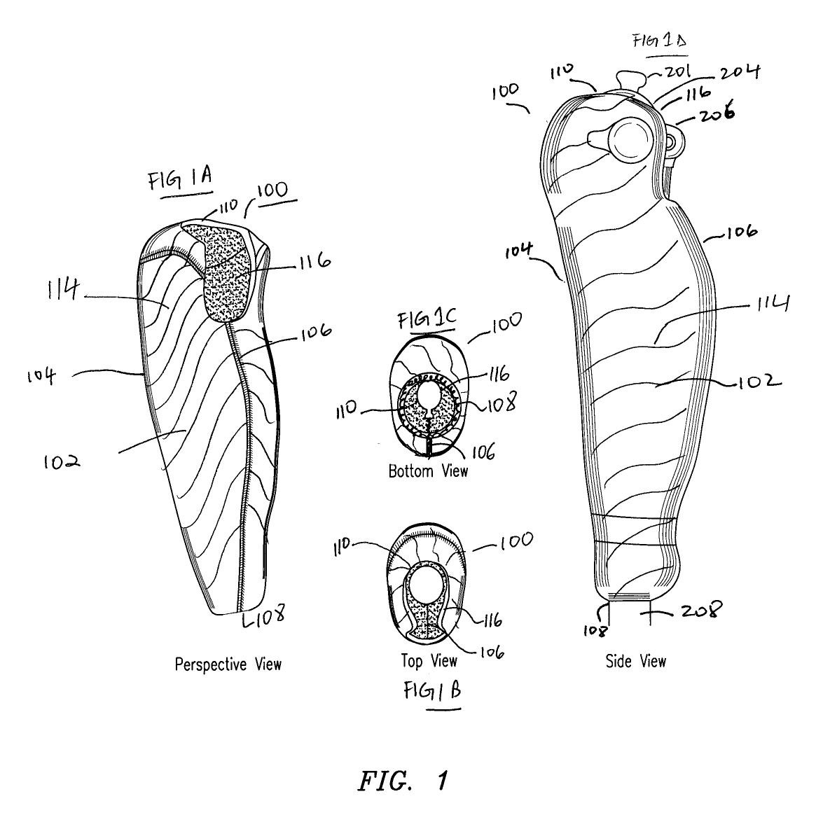

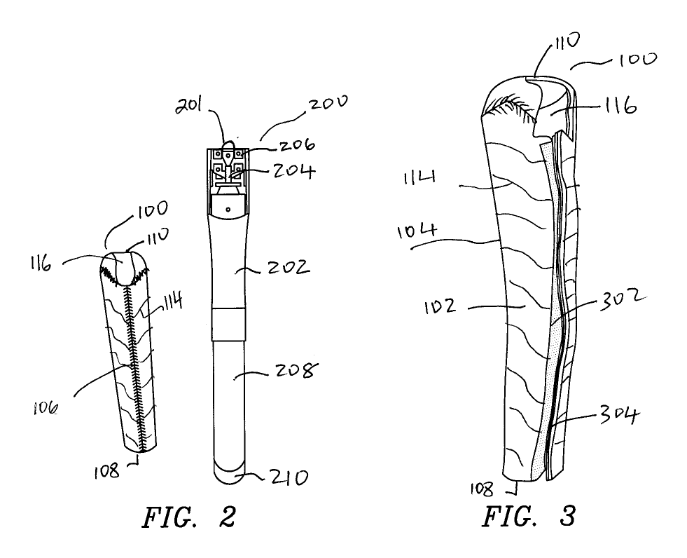

With reference now to the drawings, and in particular to FIGS. 1A-2 thereof, a novel cover for a prosthetic knee joint embodying the principles and concepts of the present invention and generally designated by the reference numeral 100 will be described. While the embodiments described herein are illustrated as an exemplary cover 100 for a prosthetic member 200 such as, for example, a prosthetic knee joint, it will be appreciated by those skilled in the art that the present invention is not limited to covers 100 for a prosthetic knee joint, and may be made in various shapes and sizes to cover and protect prosthetic members for other body parts.

Referring now to FIGS. 1A-2, an embodiment of the cover 100 for a prosthetic knee joint of the present invention will be described.

The cover 100 includes a hollow flexible body portion 102 having a generally tapered cylindrical body 104. According to an exemplary embodiment, the cylindrical body 104 has a curvilinear shape. The cover 100 further includes a seam 106 extending upwardly from a lower-end 108 to a upper-end 110. The cover 100 is specifically sized to fit around a housing 202 of the prosthetic member 200 and to allow exposure of a mechanical linkage 204 through a slotted region 116 formed in the upper-end 110 of the cover 100. According to an exemplary embodiment, the slotted region 116 is shown herein to be comprising an oval slot 116 that has a depth sufficient to allow articulation of a prosthetic knee 206 and of a width allowing maximum protection possible of the mechanical linkage 204 of a prosthetic limb 208 of the prosthetic member 200 in such a way as to allow the mechanical linkage 204 to move. It is important to note that FIGS. 1A-2 and the description herein are directed to a universal cover 100 for a prosthetic knee joint such that the cover 100 can be worn interchangeably on the left and the right prosthetic member 200 of the user.

The cover 100 for the prosthetic knee joint is flexible and can be made of various materials which are water-proof to protect the user's prosthetic knee joint from dirt, debris and/or water. In one embodiment of the present invention, the cover 100 can be made of a material that is water-proof and easy to clean such as, for example, ⅛ inch thick perforated VeLPlush™ Neoprene, which provides softness and elasticity. Although the cover 100 is disclosed as being formed of neoprene, it will be appreciated that the cover 100 according to the present invention can be formed from a variety of different materials which are flexible and water-proof. According to an exemplary embodiment, the cover 100 is formed of neoprene with an aesthetically pleasing design, having a surface 114 of camouflage pattern which has gained popularity in recent years. Multiple patterns can be used in accordance with the principles of the present invention.

Because of the neoprene construction, the cover 100 has sufficient rigidity to extend alongside the spatial region around the mechanical linkage 204 of the prosthetic limb 208 and yet flex when engaged by any moving part. The ability of the neoprene surface to both flex and protect is critical to the design of the cover 100. The specific shape, size and cut-out configuration of the cover 100 is, however, specifically adapted for the prosthetic knee 206 of the prosthetic limb 208 for affording multiple advantages for both the prosthetic member manufacturer as well as the user.

The prosthetic member manufacturer is able to design a product with the understanding that some element of protection can be afforded to the mechanical linkage 204 and the user has the added ability to enjoy outdoor activities while attempting to provide a degree of protection in an aesthetically pleasing configuration particularly adapted for protection of the prosthetic limb 208. The prosthetic limb 208 includes an upper attachment region 201 having the mechanical linkage 204 extending downwardly therefrom. The mechanical linkage 204 comprises a portion of the prosthetic knee 206 adapted for providing angular movement for a lower portion 210 of the prosthetic limb 208. The lower portion 210 is shown without an additional prosthetic member attached thereto but, in this particular example, would generally include a prosthetic foot secured thereto.

FIG. 1B illustrates a top view of the cover 100. FIG. 1B further illustrates a slotted region 116 formed in an upper portion 110 of the cover 100. According to an exemplary embodiment, the slotted region 116 is shown herein to be comprising an oval slot 116 that has a depth sufficient to allow the articulation of the prosthetic knee 206 and of a width allowing maximum protection possible of the mechanical linkage 204 of the orthopedic limb 208 of the prosthetic member 200 in such a way as to allow the mechanical linkage 204 to move.

FIG. 1C illustrates a bottom view of the cover 100. FIG. 1C further illustrates a slotted region 116 formed in an upper-end 110 of the cover 100. The cover 100 further includes a seam 106 extending upwardly from a lower-end 108 to the upper-end 110.

FIG. 1D illustrates a side view of the cover 100 placed over the prosthetic member 200. The cover 100 is specifically sized to fit around a housing 202 (FIG. 2) of the prosthetic member 200 and to allow exposure of a mechanical linkage 204 through a slotted region 116 formed in an upper portion 110 of the cover 100. The mechanical linkage 204 comprises a portion of the prosthetic knee 206 adapted for providing angular movement for a lower portion 210 of the prosthetic limb 208. According to an exemplary embodiment, the slotted region 116 has a depth sufficient to allow the articulation of a prosthetic knee 206 and of a width allowing maximum protection possible of the mechanical linkage 204 of a prosthetic limb 208 of the prosthetic member 200 in such a way as to allow the mechanical linkage 204 to move.

FIG. 2 illustrates a perspective view of the cover 100 for the prosthetic knee joint of FIG. 1A shown along side the prosthetic member 200. It may be seen that the mechanical linkage 204 extends downwardly into the prosthetic limb 208 and into an enlarged housing 202 constructed therearound. The housing 202 contains both the mechanical linkage 204 as well as other mechanical devices facilitating the actuation of the prosthetic knee 206. To further facilitate such actuation, electronic systems can be included therein and specifically programmed for the user of the prosthetic limb 208 to enhance the functioning thereof. The use of the cover 100 as described herein allows a partial covering of the exposed mechanical linkage 204 and related elements associated therewith and further helps prevent the unwanted intrusion of dirt debris and/or water into the housing 202.

In operation, the cover 100 for the prosthetic limbs of the present invention provides a protective cocoon with upper and lower openings 108, 110 adapted for receiving and providing maximum protection for an articulating member. The cover 100 according to FIGS. 1A-2 is adapted to be slipped onto the prosthetic member 200. Various surface designs and patterns may be used as well as various thicknesses of neoprene. Likewise, the present invention is not limited to the use of neoprene as other materials may prove satisfactory in their use as orthopedic covers for prosthetic limbs. The slotted region 116 as shown herein is furthermore of an elongate characteristic for affording the articulating movement herein described and other cutout shapes and clearance designs may be utilized with various limb joint configurations as necessitated by the linkage and/or prosthetic knee designs.

FIG. 3 illustrates an alternative embodiment of a cover 100 for a prosthetic knee joint according to the present invention. The cover 100 is substantially similar to the one disclosed earlier with reference to FIGS. 1A-2, however, the seam 106 extending upwardly from the lower-end 108 to the upper-end 110 as disclosed in FIG. 1A is replaced with a hook and pile surface 302 and 304 to facilitate securement of the cover about a housing 202 of the prosthetic member 200. Portions 302 and 304 of the hook and pile material are illustrated as it appears on an outer portion of the cover 100. It should be noted that the term “hook and pile fasteners” is a recognized structure to one skilled in the art and is often sold under the trademark Velcro®. It is well known that the hook and pile material engage one another. Therefore, if surface 302 as shown in FIG. 3 is a hook surface, then the surface 304 as shown on FIG. 3 is a pile surface. It is to be understood that further reference herein to a “hook and pile surface” refers to either a hook or a pile surface. This particular embodiment of the cover 100 having portions 302 and 304 of the hook and pile material allows a user to wrap the cover 100 around housing 202 of the prosthetic member 200 and secure the cover 100 on the prosthetic member 200 using the portions 302 and 304 of the hook and pile material.

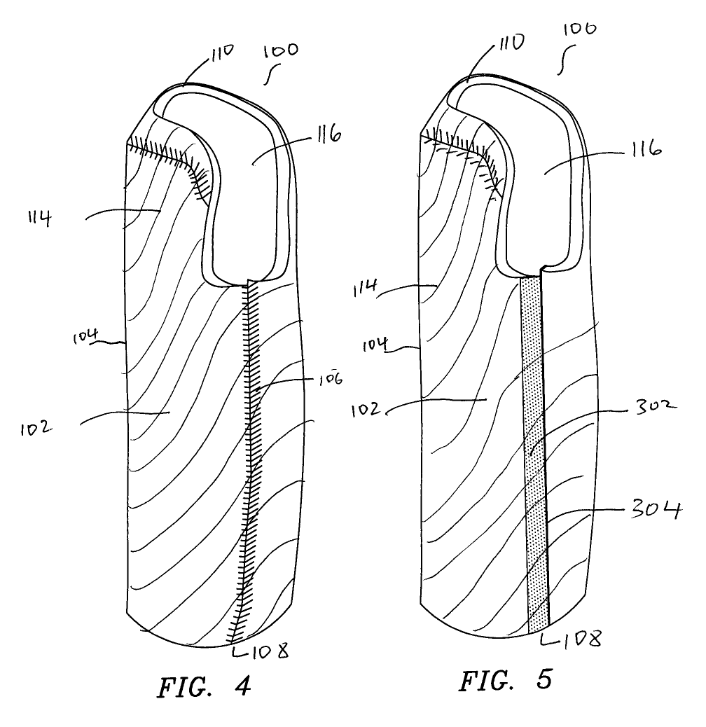

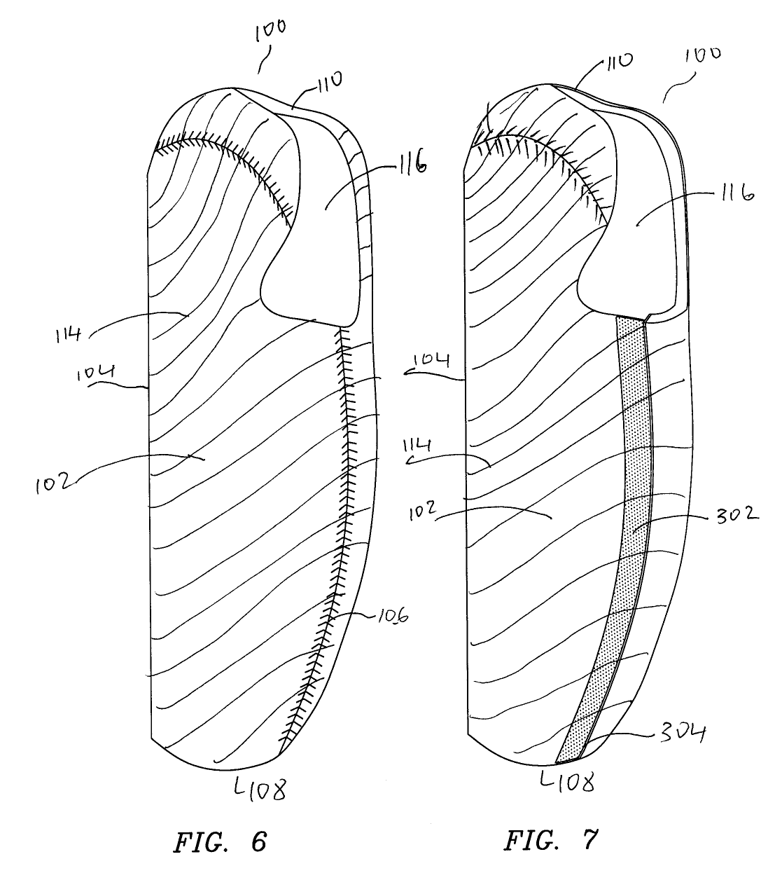

According to an embodiment of the present invention, the cover 100 includes a flexible body portion 102 having a generally tapered cylindrical body 104. However, the cover 100 embodying the novel concept as discussed above can be of any shape so long as it is sized and shaped to fit around a housing 202 of the various limb joint configurations as necessitated by the linkage and/or prosthetic member designs. As an exemplary embodiment, a few variations of cover 100 are illustrated in FIGS. 4-7 embodying the novel concept for protecting a prosthetic knee joint 200 from dirt, debris and/or water as discussed above.

Although preferred embodiments of the different kinds and shapes of a cover 100 of the present invention have been illustrated in the accompanying Drawings and described in the foregoing Detailed Description, it will be understood that the invention is not limited to the embodiments disclosed, but is capable of numerous rearrangements, modifications and substitutions without departing from the spirit of the invention as set forth and defined by the following claims.

1. An orthopedic cover for a prosthetic member comprising:
a hollow flexible body portion;
a generally tapered cylindrical body adapted for being received over the prosthetic member;
an upper opening having a depth sufficient to allow articulation of a prosthetic knee of the prosthetic member and a width sufficient to allow protection of a mechanical linkage of the prosthetic member; and
a lower opening exposing a prosthetic limb outwardly therefrom.

2. The cover according to claim 1, comprising a seam extending upwardly from the lower opening to the upper opening.

3. The cover according to claim 1, comprising a hook and pile surface to facilitate securement of the cover about a housing of the prosthetic member.

4. The cover according to claim 1, wherein the cover is made of a material that is water-proof and easy to clean.

5. The cover according to claim 4, wherein the cover is formed from Neoprene.

6. The cover according to claim 1, wherein the cover is sized to accommodate prosthetic members of various sizes and shapes.

7. The cover according to claim 1, wherein the upper opening comprises an oval shape to allow articulation of the prosthetic knee.

8. The cover according to claim 1, comprising a surface having an aesthetically pleasing design.

9. An orthopedic cover for a prosthetic member comprising:
a generally cylindrical body having a curvilinear shape and adapted for receipt of a prosthetic member therethrough;
a slotted opening in an upper end of the cover adapted to protect mechanical elements of the orthopedic member while affording a mechanical linkage portion of the prosthetic member to pass through and permit necessary movement of the prosthetic member; and
an opening in a lower end of the cover to allow an end of the prosthetic member pass through.

10. The cover according to claim 9, comprising a seam extending upwardly from the lower opening to the upper opening.

11. The cover according to claim 9, comprising a hook and pile surface to facilitate securement of the cover about a housing of the prosthetic member.

12. The cover according to claim 9, wherein the cover is made of a material that is water-proof and easy to clean.

13. The cover according to claim 12, wherein the cover is formed from Neoprene.

14. The cover according to claim 9, wherein the cover is sized to accommodate prosthetic members of various sizes and shapes.

15. The cover according to claim 9, wherein the upper opening comprises an oval shape to allow articulation of the prosthetic knee.

16. The cover according to claim 9, wherein the upper opening has a depth sufficient to allow articulation of a prosthetic knee of the prosthetic member and a width sufficient to allow protection of the mechanical linkage of the prosthetic member.

17. The cover according to claim 9, comprising a surface having an aesthetically pleasing design.

18. An orthopedic cover for a prosthetic member of a given shape comprising:
a hollow flexible body portion;
a configured cylindrical body adapted for being received over the prosthetic member for covering the shape thereof,
an upper opening having a depth sufficient to allow articulation of a prosthetic knee of the prosthetic member and a width sufficient to allow protection of a mechanical linkage of the prosthetic member;
a lower opening exposing a prosthetic limb outwardly therefrom; and
a hook and pile surface to facilitate securement of the cover about a housing of the prosthetic member.

19. The cover according to claim 18, wherein the cover is made of a material that is water-proof and easy to clean.

20. The cover according to claim 18, wherein the cover is sized to accommodate prosthetic members of various sizes and shapes.
